(19) United States
(12) Patent Application Publication
(10) Pub. No.: US 2011/0252511 A1
(43) Pub. Date: Oct. 13, 2011

(54) Method For Colouring A Flower On A Pot Plant, And Pot Plant With Coloured Flower

(75) Inventor: Jeffrey Franciscus VAN DEN BERGHEN, Naaldwijk (NL)

(73) Assignees: R.C.G. DE KONING HOLDINGS B.V., Honselersdijk (NL); LOGICO B.V.,, Honselersdijk (NL)

(21) Appl. No.: 12/901,183

(22) Filed: Oct. 8, 2010

(30) Foreign Application Priority Data
Oct. 12, 2009 (NL) NL-2003621

(51) Int. Cl. A01H 5/00; A01G 5/00

(52) U.S. Cl. 800/323; 47/57.5

(57) ABSTRACT

A method for colouring a flower on a pot plant comprises the steps of (i) providing a pipette (8), (ii) arranging in the stem of the flower a bore with a diameter corresponding to the diameter of the outer end of the pipette, (iii) inserting the outer end of the pipette into the bore and fixing the pipette relative to the stem, (iv) filling the pipette with a quantity of colouring liquid, which is non-toxic to the pot plant, following any of the steps (i), (ii) or (iii) as desired, and (v) removing the pipette after the quantity of colouring liquid has been taken up by the pot plant, and pot plant with at least one flower which is coloured with a colouring liquid introduced via the stem of this flower.

This application claims the priority of Netherlands Application No. NL 2003621, filed Oct. 12, 2009, which is herein incorporated by reference in its entirety.

### BACKGROUND

1. Field of the Invention

The present invention relates generally to a method for colouring a flower on a pot plant.

2. Background of the Invention

It is known to colour cut flowers by placing a cut branch in water in which a dye has been dissolved or by spraying flowers with a paint substance.

It is further known to colour the flowers of a pot plant by dissolving a dye in water which is then poured onto the soil around the roots of the plant and is taken up by the roots.

These known methods have the drawback that they are undesirable for logistical reasons (placing cut branches in a dye solution), produce an aesthetically unsatisfactory result (spraying with a paint substance) or have a low efficiency (administering a dye solution around the roots).

Known from WO 93/02546 is a method for introducing liquids, such as pesticides, fungicides, nutrients, water, flavorants, odorants, colorants or suitable solutions of such materials into a plant. In order to perform the known method a device is provided which comprises a liquid reservoir and spikes connected thereto via a manifold, which spikes, which are each provided with a T-shaped passageway, are pressed into the stem or trunk of a plant or tree in a manner such that the transverse parts of the T-shaped passageway debouch into the xylem system of the plant or tree. After arranging the spikes in the plant stem or tree trunk the apparatus is attached to the plant or tree, and liquid is admitted from the reservoir into the xylem system of the plant via the passages in the respective spikes.

The known device is relatively heavy, has a complex construction and, as a result of this complexity, is expensive to produce and is for these reasons unsuitable for use to colour the flowers on a pot plant.

### BRIEF SUMMARY OF THE INVENTION

It is an object of the invention to provide a method for colouring a flower on a pot plant which can be performed in relatively simple manner, wherein a dye is administered with a high efficiency and which can be performed at relatively low cost without the use of complex equipment.

This object is achieved, and other advantages gained, with a method which according to the invention comprises the steps of (i) providing a pipette, (ii) arranging in the stem of the flower a bore with a diameter corresponding to the outer diameter of the outer end of the pipette, (iii) inserting the outer end of the pipette into the bore and fixing the pipette relative to the stem, (iv) filling the pipette with a quantity of colouring liquid, which is non-toxic to the pot plant, following any of the steps (i), (ii) or (iii) as desired, and (v) removing the pipette after the quantity of colouring liquid has been taken up by the pot plant.

### BRIEF DESCRIPTION OF THE DRAWINGS

FIG. 1 is a perspective view of a pot with a plant having a stem and flowers with a pipette in accordance with an embodiment of the present invention.

FIG. 2 is a detail perspective view of a plant stem and pipette in accordance with an embodiment of the present invention.

### DETAILED DESCRIPTION OF THE INVENTION

The invention will be elucidated hereinbelow on the basis of an exemplary embodiment, with reference to the drawings.

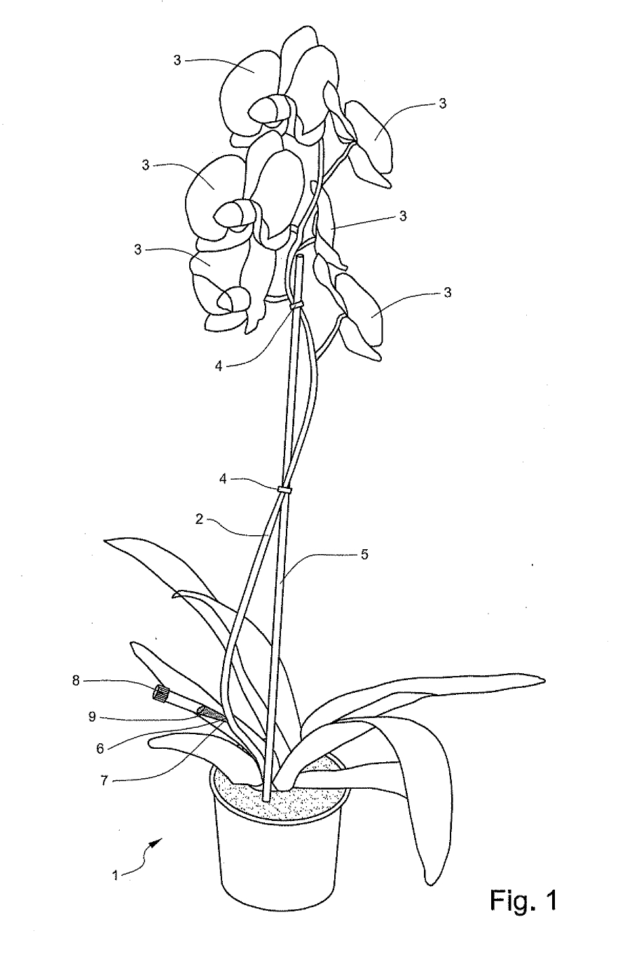

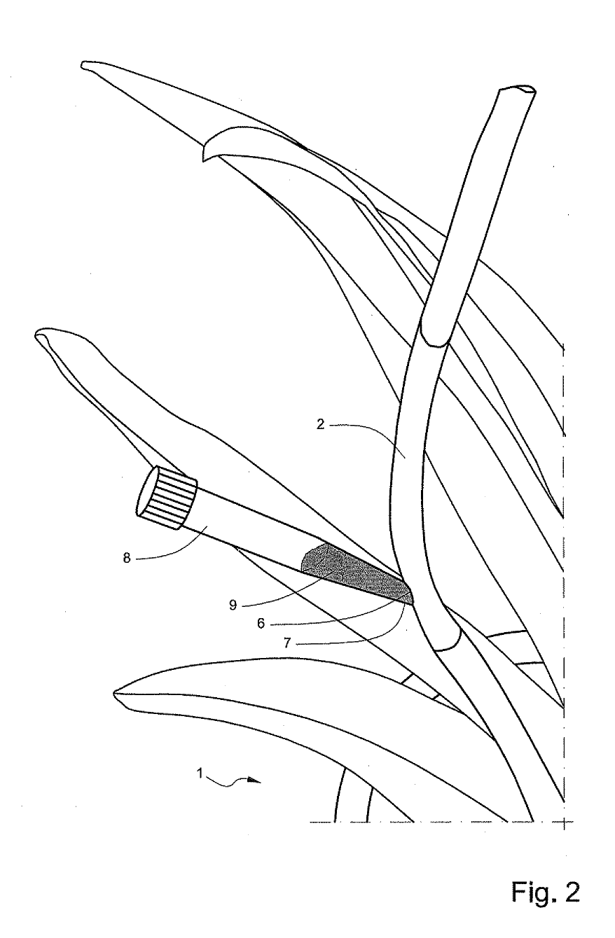

In the drawings, FIG. 1 shows a perspective view of a pot 1 with a white phalaenopsis with a flower stem 2 and flowers 3. Using an adhesive strip 4 stem 2 is adhered at two locations to a pin 5 placed in the pot. After the plant has been given sufficient water, which has been drawn up by the plant, a hole 6 is drilled into the lower side of stem 2 (shown in detail in FIG. 2), into which is inserted the outer end 7 of a pipette 8 filled with colouring liquid 9 comprising blue triphenylmethane dye. Pipette 8 is not attached to pin 5 but hangs completely freely with its outer end 7 in flower stem 2 and, after the colouring liquid has been taken up, can be removed in simple manner with a rotating and pulling movement. It has been found that after a period of about 12 hours, in which the colouring liquid is taken up, flowers 3 take on a blue appearance in the centre and along the edges.

In accordance with an embodiment of the present invention, a method for colouring a flower includes the steps of: (i) providing a pipette, (ii) arranging in the stem of the flower a bore with a diameter corresponding to the outer diameter of the outer end of the pipette, (iii) inserting the outer end of the pipette into the bore and fixing the pipette relative to the stem, (iv) filling the pipette with a quantity of colouring liquid, which is non-toxic to the pot plant, following any of the steps (i), (ii) or (iii) as desired, and (v) removing the pipette after the quantity of colouring liquid has been taken up by the pot plant.

Colouring a flower of a pot plant according to the method of the present invention has an advantage over the known method that use is made of commercially available pipettes instead of a complicated apparatus developed for this purpose.

It has moreover been found that, in order to fix the pipette relative to the stem as according to step (iii), it suffices in practical situations to insert the pipette with its outer end in the arranged bore. Because the diameter of the bore corresponds to the outer diameter of the outer end of the pipette, and the stem of the flower provides sufficient depth and strength, a pipette with its outer end inserted into the bore remains in place without additional measures being required for this purpose.

The step (ii) of arranging a bore is preferably preceded by administering water to the plant.

The colouring liquid is taken up spontaneously by the flower by means of the rising sap flow in the flower stem, wherein a period of at least 8 hours, preferably about 12 hours, suffices to allow the flower to take up as much colouring liquid that it takes on the desired colour.

Any dye which is non-toxic for the pot plant and water-soluble can in principle be applied.

In an exemplary embodiment of a method according to the invention the colouring liquid comprises an organic dye.

The organic dye comprises for instance a triphenylmethane dye.

The method is in principle suitable for application to numerous pot plants. The method is suitable, for example, for colouring the flower on a pot plant selected from the group of plant genera comprising phalaenopsis, anthurium and spatyfillum.

The invention also relates to a pot plant with at least one flower which is coloured with a colouring liquid introduced via the stem of this flower, which pot plant is for instance selected from the group of plant genera comprising phalaenopsis, anthurium and spatyfillum.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A method for colouring a flower on a pot plant, comprising the steps of
(i) providing a pipette;
(ii) arranging in a stem of the flower a bore with a diameter corresponding to the diameter of an outer end of the pipette;
(iii) inserting the outer end of the pipette into the bore and fixing the pipette relative to the stem;
(iv) filling the pipette with a quantity of colouring liquid, which is non-toxic to the pot plant, following any of the steps (i), (ii) or (iii) as desired, and
(v) removing the pipette after the quantity of colouring liquid has been taken up by the pot plant.

2. The method as claimed in claim 1, wherein the step (ii) of arranging a bore is preceded by administering water to the plant.

3. The method as claimed in claim 1, wherein the removal step (v) is performed after a period of at least 8 hours, during which the pipette filled with the colouring liquid has been inserted with its outer end in the bore.

4. The method as claimed in claim 3, wherein the period is about 12 hours.

5. The method as claimed in claim 1, wherein the colouring liquid comprises an organic dye.

6. The method as claimed in claim 5, wherein the organic dye comprises a triphenylmethane dye.

7. The method as claimed in claim 6, wherein the triphenylmethane dye is a blue dye.

8. The method as claimed in claim 1, wherein the pot plant is selected from the group of plant genera consisting of phalaenopsis, anthurium and spatyfillum.

9. A pot plant with at least one flower which is coloured with a colouring liquid introduced via the stem of the at least one flower.

10. The pot plant as claimed in claim 9, selected from the group of plant genera consisting of phalaenopsis, anthurium and spatyfillum.
